(19) United States
(12) Patent Application Publication
(10) Pub. No.: US 2011/0022091 A1
(43) Pub. Date: Jan. 27, 2011

(54) LOCAL DELIVERY OF AN ACTIVE AGENT FROM AN ORTHOPEDIC IMPLANT

(75) Inventors: Kent M. Anderson, Mountain View, CA (US); Matthew M. Morrison, Cordova, TN (US); Jonathan Dewey, Raleigh, NC (US); Aurelien Bruneau, Jacksonville, FL (US); Fred J. Molz, IV, Birmingham, AL (US); Thomas Carls, Memphis, TN (US); Eric C. Lange, Pleasanton, CA (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/896,169

(22) Filed: Oct. 1, 2010

Related U.S. Application Data
(62) Division of application No. 11/117,891, filed on Apr. 29, 2005.

(51) Int. Cl. A61B 17/70; A61B 17/88; A61M 31/00

(52) U.S. Cl. 606/249; 606/279; 604/93.01

(57) ABSTRACT

A posterior spinal fixation device or dynamic spinal stabilization device or other orthopedic device includes an active agent-delivery component. The active agent-delivery component has an active agent impregnated therein or adsorbed thereon or otherwise contained therein and is configured to release the active agent locally after the device is implanted in a patient. One preferred type of implant in accordance with the invention is an implant for stabilizing a spinal motion segment that includes a spacer member positionable between adjacent spinous processes or transverse processes or other posterior spinal element, including an implanted anchor element.

This application is a divisional of co-pending U.S. patent application Ser. No. 11/117,891, filed Apr. 29, 2005 which is incorporated herein by reference in its entirety.

### BACKGROUND

A wide variety of orthopedic implant devices are known that are designed to be affixed to posterior vertebral elements for providing structural support to a patient's spine. Implants can be positioned between adjacent spinous processes to provide resistance to vertebral movement as a result of extension of the spinal column. These implants can provide a shock absorber or bumper that dynamically limits spinal extension. The implants can also be secured to the adjacent spinous processes with looped cables or straps that extend completely about the spinous processes and implant to maintain positioning of the implant between the spinous processes while also limiting spinal flexion to provide dynamic stabilization along the spinal midline. They can alternatively be held in place by other means, such as, for example, by tethers affixed to other spinal elements. Other implants can be configured for placement between transverse processes of adjacent vertebrae or between other posterior spinal elements to provide dynamic stabilization at uni-lateral or bi-lateral locations of the posterior vertebral elements. In addition to dynamic spinal stabilization devices, a wide variety of other types of posterior vertebral appliances are known for use in rigid posterior spinal fixation systems, such as rods, plates, tethers and staples, for example.

As with any surgical procedure, to facilitate proper healing after surgical implantation of orthopedic implant devices, one or more therapeutic active agents, such as, for example, anti-inflammatory agents, analgesic agents, anti-microbial or anti-viral agents, and the like are administered to the patient. However, systemic administration of many types of active agents can have harmful effects or otherwise be undesirable. Furthermore, alternative therapeutic agents could be selected for administration to a post-operative patient that would otherwise be desirable were it not for undesirable effects associated with systemic administration thereof. Thus, there is a need for innovation in the way that post-operative therapeutic agents are delivered to a patient after surgical implantation of an orthopedic implant device. The present invention addresses this need.

### SUMMARY

The present invention provides a variety of orthopedic implant devices that include at least one structural component and at least one component effective to deliver an active agent to the patient locally at the site of the implant. In one aspect of the invention, there is provided an orthopedic implant device including at least one structural component configured to provide structural support to one or more bones or joints; at least one active agent-delivery component; and an active agent impregnated in or adsorbed on or otherwise contained in said at least one active agent-delivery component. The implant device is configured to release the active agent locally after the implant device is implanted in a patient. In one embodiment, the active agent-delivery component comprises an absorbent or adsorbent or biodegradable material. In another embodiment, the active agent-delivery component comprises a micromechanical machine.

An exemplary orthopedic implant in accordance with the invention is a dynamic spinal stabilization device that includes a spacer member extending between opposite first and second ends and that includes a component for locally delivering an active agent. The spacer member is positionable between adjacent upper and lower spinous processes of a spinal motion segment. The active agent-delivery component can be an integral part of the spacer member or a separate component. In one embodiment, the spacer member includes a compressible body to dynamically limit movement of the upper and lower spinous processes toward one another upon extension of the spinal motion segment.

In another embodiment, the spacer member is rigid. An upper engaging member and a lower engaging member each extend from the spacer member and are engageable with the spinal motion segment to limit flexion of the spinal motion segment.

In one exemplary preferred embodiment, at least one of the upper and lower engaging members is a tether, such as, for example, a cable or strap, that is structured for positioning about the upper or lower spinous processes, respectively, and for being crimped around the spacer or to the spacer. The engaging members contact the respective spinous processes to limit flexion of the spinal motion segment. In another embodiment, at least one of the upper and lower engaging members is structured for positioning along a surface of a lamina adjacent a respective one of the upper and lower spinous processes. In this embodiment, for example, the upper engaging member can include a hook end portion positionable along a superior surface of an upper lamina adjacent the upper spinous process and the lower engaging member can include a hook end portion positionable along an inferior surface of the lower spinous process.

Another exemplary orthopedic implant in accordance with the invention is a spinal implant that includes at least two anchor members, such as pedicle screws, configured to be affixed to adjacent vertebrae; and a spacer member extending between the anchor members. In one preferred embodiment, the spacer member includes a flexible and/or compressible body sized and shaped to extend between the anchor members to dynamically limit movement of the anchor members toward one another upon extension of the spinal motion segment, and also includes a component for locally delivering an active agent. The anchor members and the spacer member can also define apertures therethrough for receiving a tether or a rod, i.e., a rigid rod or a flexible rod, as is well known in the art. Alternatively, the spacer member can be positioned within a sheath, which passes through apertures defined in the anchor members. In another embodiment, the spacer member can be a rigid spacer member. As with the interspinous implant described above, the active agent-delivery component can be an integral part of the spacer member or a separate component.

Another exemplary orthopedic implant in accordance with the invention is a spinal implant that includes a spacer member extending between opposite upper and lower ends, the upper and lower ends each including a pair of arms, and a recessed surface between the pair of arms, the arms structured to receive a respective adjacent one of upper and lower transverse processes of a spinal motion segment. In one embodiment, the spacer member includes a compressible body sized and shaped to extend between the upper and lower transverse processes to dynamically limit movement of the upper and lower transverse processes toward one another upon extension of the spinal motion segment, and also includes a component for locally delivering an active agent. In another embodiment, the spacer member can be rigid. As with the interspinous implant described above, the active agent-delivery component can be an integral part of the spacer member or a separate component. A spinal implant system can include a first spacer member extending between opposite upper and lower ends structured to receive a respective adjacent one of upper and lower transverse processes of a spinal motion segment at a first side of the spinal midline, and a second spacer member extending between opposite upper and lower ends structured to receive a respective adjacent one of upper and lower transverse processes of a spinal motion segment at a second side of the spinal midline. In this embodiment, each of the spacer members preferably includes a compressible body sized and shaped to extend between the upper and lower transverse processes to dynamically limit movement of the upper and lower transverse processes toward one another upon extension of the spinal motion segment.

In one aspect of the invention, an orthopedic implant device, or a spacer member or other component of an implant device, includes an internal structural component contained within an outer sheath. An active agent-delivery component that includes an absorbent or adsorbent or biodegradable layer can be positioned between the internal structural component and the outer sheath or on the external side of the outer sheath, or impregnated in the outer sheath material. The sheath can be, for example, a porous or permeable fabric or mesh, or an impermeable material. For example, a posterior spinal dynamic stabilization device, or a spacer member therefor, that is configured to be positioned between adjacent spinous processes or adjacent transverse processes, can comprise an inner silicone core wrapped in a woven polyester fabric. In such a device, an active agent-delivery component can be positioned between the silicone core and the polyester fabric or on the exterior surface of the fabric, or an active agent can be impregnated in the fabric itself.

Other orthopedic implant devices that are contemplated by the invention include, without limitation, posterior vertebral appliances for use in rigid posterior spinal fixation systems, such as, for example, rods, plates, tethers and staples; and bone stabilization members positionable along adjacent bone portions outside an interspace between the bone portions, such as, for example, bone plates and artificial ligaments. Such bone stabilization members find advantageous use, for example, for stabilization of joints, such as hip or knee joints. Such devices can include an active agent-delivery component formed as an integral part of the appliance or as a separate layer or component.

In one aspect of the invention, an active agent-delivery layer is affixed to at least a portion of the exterior surface of an orthopedic implant device. The active agent-delivery layer or component in alternative embodiments can comprise a biodegradable matrix material having an active agent dispersed therein that releases the active agent upon degradation or erosion of the matrix after implantation of the device, or a porous structure that releases an active agent by wicking action or other action without being degraded in situ, or an adsorbent material that releases an active agent from the surface of the component. In addition, the active agent-delivery layer or component can be formed of a rigid material or of an elastic material in various alternative embodiments of the invention.

In another aspect, the invention provides an orthopedic implant device that defines at least one aperture, and an active agent delivery component is configured to be positioned in the aperture as an insert. The insertt in alternative embodiments can comprise a biodegradable matrix material having an active agent dispersed therein, that releases the active agent upon degradation of the matrix after implantation of the device; a porous structure that releases an active agent by wicking action or other action without being degraded in situ; an adsorbent material that releases an active agent from the surface of the component; or a micromechanical machine configured for controlled release of one or more active agents. In addition, where the active agent-delivery component is of the biodegradable, porous or adsorbent type, it can be formed of a rigid material or of an elastic material in various alternative embodiments of the invention.

In yet another aspect of the invention, there is provided a posterior spinal fixation device or dynamic spinal stabilization device including an active agent-delivery component that comprises an elastic material having the active agent absorbed therein or adsorbed thereon. In one embodiment, the device is configured such that, after implantation of the device, a dose of the active agent is caused to be released at an increased rate by compressing the active agent-delivery component, or by stretching the active agent-delivery component, or by applying a torque to the active agent-delivery component. The compression, stretching, and/or torque can be exerted upon the active agent-delivery component alter implantation of the device by vertebral movement as a result of extension of the spinal column, flexion of the spinal column, bending of the spinal column or rotation of the spinal column.

In a further aspect of the invention, there is provided a method for delivering an active agent to a patient at a location adjacent an orthopedic implant device. The method includes (1) providing an orthopedic implant device comprising an active agent-delivery component, the active agent-delivery component having an active agent impregnated therein or adsorbed thereon or otherwise contained therein and configured to release the active agent locally after the device is implanted in a patient; and (2) surgically implanting the device in a posterior spinal location. The active agent-delivery component can include an elastic material having the active agent absorbed therein or adsorbed thereon. In an embodiment having an active agent-delivery component comprising an elastic material, the method can further include, after the implanting, causing a dose of the active agent to be released or released at an increased rate by (a) compressing the active agent-delivery component, (b) stretching the active agent-delivery component, or (c) applying a torque to the active agent-delivery component.

These and other aspects will be discussed further below.

### BRIEF DESCRIPTION OF THE DRAWINGS

FIG. 1 is a perspective view of a posterior portion of spinal column motion segment with an implant assembly engaged thereto.

FIG. 2 is a cross-sectional view of one embodiment of the spinal motion segment of FIG. 1 showing structure of a first orthopedic implant device of the invention.

FIG. 3 is a cross-sectional view of another embodiment of the spinal motion segment of FIG. 1 showing structure of a second orthopedic implant device of the invention.

FIG. 4 is a cross-sectional view of yet another embodiment of the spinal motion segment of FIG. 1 showing structure of a third orthopedic implant device of the invention.

FIG. 5 is an elevation view of another embodiment implant assembly.

FIG. 6 is a perspective view of a posterior portion of spinal column motion segment with an implant assembly engaged thereto.

FIG. 7 is an elevation view of another embodiment implant assembly.

FIG. 8 is an elevation view of a posterior portion of a spinal column motion segment with implant assemblies engaged thereto.

FIG. 9 is a lateral view of the spinal column motion segment of FIG. 8.

FIG. 10 is an elevation view of another embodiment implant assembly.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

Posterior spinal implant devices are provided in one aspect of the present invention that, in addition to providing structural functionality, also function to deliver one or more active agent to tissues adjacent or near the site of the implant. In one preferred embodiment, the implant device includes an active agent-delivery component in addition to one or more structural components of the device. In another preferred embodiment, one or more of the structural components themselves have an active agent impregnated therein or adsorbed thereto for local release to a patient after surgical placement of the device.

As used herein, the term “active agent” means a substance having a therapeutic effect on the patient. Non-limiting examples of broad categories of useful active agents that can be used in accordance with the present invention are those included within the following categories: anabolic agents, anti-coagulants, anti-infective agents, anti-inflammatory agents, anti-neoplastic agents, anti-pyretic and analgesic agents, anti-spasmodic agents, anti-thrombotic agents, antihistamines, biologicals, such as bone morphogenetic proteins, diagnostic agents, neuromuscular drugs, nutritional agents, vasodilators, and pro-drugs. Examples of these and other active agents suitable for use in connection with the invention are well know to persons of ordinary skill in the art, and many are available in the literature. Representative examples are set forth in U.S. Pat. No. 6,419,709 to Mao et al., which is hereby incorporated by reference herein.

Active agents can be in different forms, such as uncharged molecules, components of molecular complexes, or non-irritating, pharmacologically acceptable salts such as hydrochloride, hydrobromide, sulphate, phosphate, nitrate, borate, acetate, maleate, tartrate, salicylate, etc. For acidic drugs, salts of metals, amines, or organic cations (e.g. quaternary ammonium) can be employed. Furthermore, simple derivatives of the drugs (such as ethers, esters, amides, etc.) which have desirable retention and release characteristics but which are easily hydrolyzed by body pH, enzymes, etc., can be employed.

The invention provides orthopedic implant devices that comprise at least one structural component configured to provide structural support to one or more bones or joints, at least one active agent-delivery component; and an active agent impregnated in or adsorbed on or otherwise contained within said at least one active agent-delivery component. In one embodiment, the active agent-delivery component comprises an absorbent or adsorbent or biodegradable material. The implant device is configured to release the active agent locally after the implant device is implanted in a patient. The active agent is, therefore, released only at the site where it is desired, i.e., where the prosthetic article is positioned.

As used herein, the term “absorbent” is used to refer to a solid object or component in the form of a porous matrix that defines internal interconnections, channels, voids and recesses, and that is effective to take in and contain a second substance (i.e., an active agent) and release the second substance when conditions permit. For example, the second substance can be released via a wicking action or other flowing action resulting from the passage of a fluid past or through the pores, channels, voids and/or recesses or release can result from a squeezing, stretching or torquing action exerted upon the absorbent object or component that causes compression of all or a portion of the absorbent object forcing the second substance from the voids and recesses through one or more pores. It is of course understood that, in an embodiment in which the porous matrix is rigid, or substantially rigid, and non-biodegradable, release of the active agent will typically result from water diffusing into the matrix, dissolving the active agent, and diffusing or wicking the active agent through the channels, voids and recesses and out of the component through the pores. In an embodiment in which the matrix is elastic and non-biodegradable, the active agent can be released in the same manner, or release can be accelerated by compression, stretching or torquing of the matrix, which squeezes active agent from the voids, recesses and channels of the matrix.

The term “adsorbent” is used herein to refer to an object or component that is capable of attaching and accumulating other substances to its surface without any chemical action. As it relates to the present invention, it is contemplated that an object or component having an active agent adsorbed thereon would hold the active agent to its surface prior to implantation of the device, and then release the active agent after implantation of the device, thereby resulting in local delivery of the active agent. It is also contemplated that the release of the active agent will typically occur without chemical alteration of the underlying surface or of the active agent.

The term “biodegradable” refers to an object or component that is capable of being decomposed into innocuous products by biological agents or otherwise eroded under the conditions present in the environment in which the device is placed during surgery. As it relates to the present invention, a biodegradable component is contemplated that includes an active agent seeded, embedded or otherwise dispersed therein, such that, as the component is decomposed or eroded after implantation of the device, the active agent is released, thereby resulting in local delivery of the active agent. The biodegradable matrix, or carrier, can comprise, for example, a biodegradable polymer or a biodegradable ceramic.

As used herein, the term “impregnated” refers to a relationship between two materials whereby one material is completely or partially filled, or saturated, with the other. Thus, the term “impregnated” can refer to an absorbent material that has an active agent absorbed therein, or to a biodegradable material having an active agent seeded, embedded or otherwise dispersed therein.

When the wording “absorbent or adsorbent or biodegradable” or like wording is used herein, such wording is intended to refer to any one of the named features or any combination of the features. For example, this wording is intended to refer to an object or component that is absorbent and biodegradable, an object or component that is adsorbent and biodegradable, an object or component that is absorbent and adsorbent, or an object or component that is absorbent, adsorbent and biodegradable.

Certain implants are positionable between posterior spinal elements, such as, for example, adjacent spinous processes of a spinal motion segment and/or between adjacent transverse processes to rigidly or dynamically stabilize and limit extension, flexion, bending and/or rotation movements of the spinal column. In one exemplary implant system for dynamic stabilization, the implant includes a spacer member received between the spinous processes that is compressible to allow extension motion of the motion segment while maintaining a distraction force between the spinous processes. The implant further includes engaging members extending from each of the upper and lower ends of the spacer member. The engaging members engage the spinal motion segment to limit flexion. In one representative embodiment of the invention, such an interspinous dynamic stabilization device is provided that is configured for local delivery of an active agent in accordance with the invention.

The engaging members can have a wide variety of configurations. In one representative interspinous dynamic stabilization system, the engaging members are tethers, such as, for example, cables or straps, configured to be fastened around the spinous processes to hold the spacer member in position. In another representative system, at least one of the engaging members is structured to engage a surface of the lamina adjacent the respective spinous process. The lamina provides a stable support surface suited to resisting loads applied thereto by the implant in resisting flexion of the motion segment. Engagement of the lamina with the engaging member also reduces torsional loading on the posterior vertebral elements. In another embodiment, each of the upper and lower engaging members of the implant assembly is engageable along a surface of a lamina adjacent the respective spinous process. The engaging members engage surfaces of the lamina opposite the surfaces of the spinous process supported by the respective end of the spacer member. In a further embodiment, the engaging members include hooked ends, and the hooked end of the upper engaging member extends along the superior surface of the upper lamina and the hooked end of the lower engaging member extends along the inferior surface of the lower member. In another embodiment, the engaging members are movably coupled with the spacer member. In yet another embodiment, at least one of the upper and lower engaging members includes a resilient connecting portion allowing limited flexion of the motion segment while maintain engagement of the engaging member with the lamina.

Other representative interspinous dynamic stabilization systems are described in U.S. Pat. No. 6,626,944 to Taylor; U.S. Patent Application Publication No. 2004/0049190; and U.S. Patent Application Publication No. 2004/0002708, each of which is hereby incorporated herein by reference in its entirety.

In another representative example of a posterior spinal implant device that can be configured to locally deliver an active agent in accordance with the invention, the implant device is an anchor-based system, such as a pedicle screw-based system. In a pedicle screw-based system, pedicle screws are inserted into adjacent vertebrae in a manner whereby a rod or cable or other structure can be affixed thereto, to provide structural support to the subject motion segment. A person of ordinary skill in the art will appreciate that a dynamic stabilization system can include a flexible rod or a cable affixed to the pedicle screws, and a rigid fixation system can be provided by connecting the pedicle screws to a rigid rod. Such a system can be configured to deliver an active agent, for example, by coating one or more components of the system with an active agent delivery coating, by inserting an active agent delivery component into an aperture formed in a component of the system, or by positioning a compressible spacer element comprising an active agent delivery component between anchoring members.

In yet another exemplary posterior spinal implant device that can be configured for active agent delivery in accordance with the invention, the implant device includes a spacer member received between the transverse processes that is compressible to allow extension motion of the motion segment while maintaining a distraction force between the transverse processes. In addition, spacer members can be positioned bi-laterally relative to a spinal motion segment in order to provide bi-lateral stabilization. In another implant system, unilateral stabilization is provided by the implant system. In still other systems, multi-level vertebral stabilization is contemplated for either uni-lateral or bi-lateral systems. One or more of the stabilization devices in such a system can be configured to deliver an active agent in accordance with the invention. The implant systems may be employed either alone or in combination with other implants, such as rods, plates, tethers, interbody fusion devices, interbody spacers, artificial discs, annulus repair system, or staples, for example. As with interspinous dynamic stabilization devices, one or more engaging members in the form of a cable or tether is typically used to couple the implant to one or more posterior vertebral elements or implants. The engaging member or members can be engaged to the spacer member, or extend through the spacer member. The engaging members can be engaged to the posterior elements in a configuration that limits spinal flexion, or simply in a manner that prevents the spacer member from being displaced from its implantation location between the transverse processes.

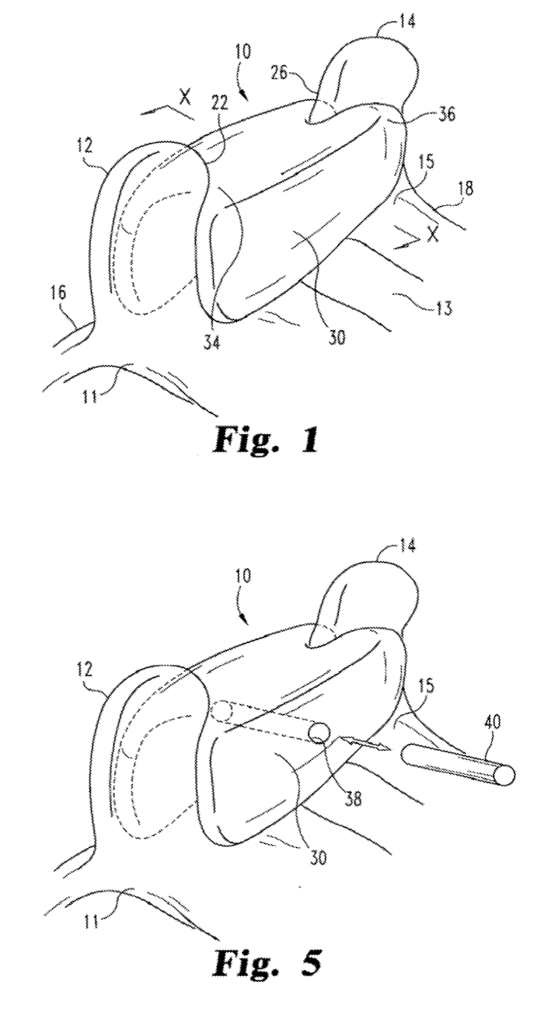

Referring now to the drawings, depicted in FIG. 1 is an inter-spinous dynamic fixation device 30, which is but one example of a type of posterior spinal implant that can be configured to deliver an active agent as contemplated by the invention, and thus is one preferred form of the invention. In FIG. 1 there is shown a spinal column segment 10 including an upper vertebra 11, a lower vertebra 15 and a spinal disc 13 therebetween. The vertebrae 11, 15 and disc 13 comprise a spinal motion segment, it being understood that a spinal motion segment may include multiple vertebral levels. Upper vertebra 11 includes an upper spinous process 12 extending from an upper lamina 16. Lower vertebra 15 includes a lower spinous process 14 extending from a lower lamina 18. The spinous processes 12, 14 and laminae 16, 18 comprise posterior elements of the vertebrae of the spinal motion segment.

Spinal implant device 30 is positioned in engagement with the posterior vertebral elements to provide dynamic spinal stabilization. Spinal implant device 30 is a spacer member extending between and contacting adjacent surfaces of spinous processes 12, 14 to limit movement of the spinous processes toward one another as a result of extension of the spinal motion segment. For example, device 30 can include an upper end 34 in contact with inferior surface 22 of spinous process 12, and a lower end 36 in contact with superior surface 26 of spinous process 14. Device 30 can include a body structured to resiliently compress in response to extension of the spinal motion segment, providing resistance to the extension forces and limiting movement of the spinous processes 12, 14 toward one another as device 30 is compressed. Implant device 30 can be affixed to vertebra 11 and vertebra 15 in any suitable manner, many alternatives of which are known in the art, and a few of which are discussed herein.

Device 30 can be fabricated from one or more components that are flexible or exhibit at least some flexibility. Examples of such components include woven fabric tubing, woven and non-woven mesh, or braided or woven structures, sutures, tethers, cords, planar members, bands, wires, cables, or any other component capable of extending between and supporting the adjacent spinous processes. In certain preferred embodiments, device 30 is fabricated from one or more components that are elastic, and is itself elastic, so it can assume various shapes during and after insertion and attachment. As used herein, the term “elastic” refers to a physical characteristic of a material whereby it is capable of being compressed, stretched or twisted, and capable of resuming its original shape after being compressed, stretched or twisted.

Device 30 can be made from any biocompatible material, material of synthetic or natural origin, and material of a resorbable or non-resorbable nature. Suitable examples of spacer member material include autograft, allograft or xenograft; tissue materials including soft tissues, connective tissues, demineralized bone matrix and combinations thereof; resorbable materials including polylactide, polyglycolide, tyrosine-derived polycarbonate, polyanhydride, polyorthoester, polyphosphazene, calcium phosphate, hydroxyapatite, bioactive glass, collagen, albumin, fibrinogen and combinations thereof; and non-resorbable materials including polyethylene, polyester, polyvinyl alcohol, polyacrylonitrile, polyamide, polytetrafluorethylene, poly-paraphenylene terephthalamide, polyetheretherketone, cellulose, titanium, silicone and combinations thereof.

Device 30 can be manufactured of a uniform composition, or can be formed using multiple diverse materials. It is of course understood that device 30 would be formed of one or more compressible materials where it is desired for the device to be used in an application where it is desirable for device 30 to be compressible. In one preferred embodiment, device 30 has an exterior surface and an active agent-delivery component layer is affixed to at least a portion of said exterior surface. Active agent-delivery layer can be formed on the surface of device 30 in a wide variety of ways known in the art.

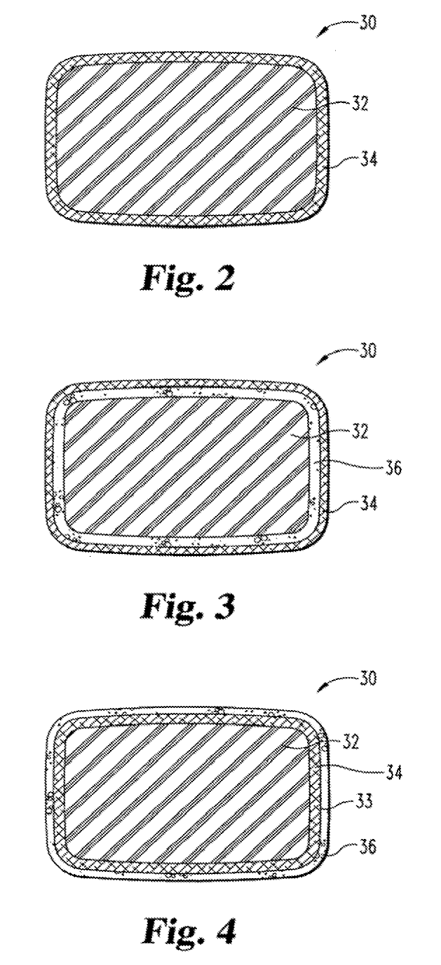

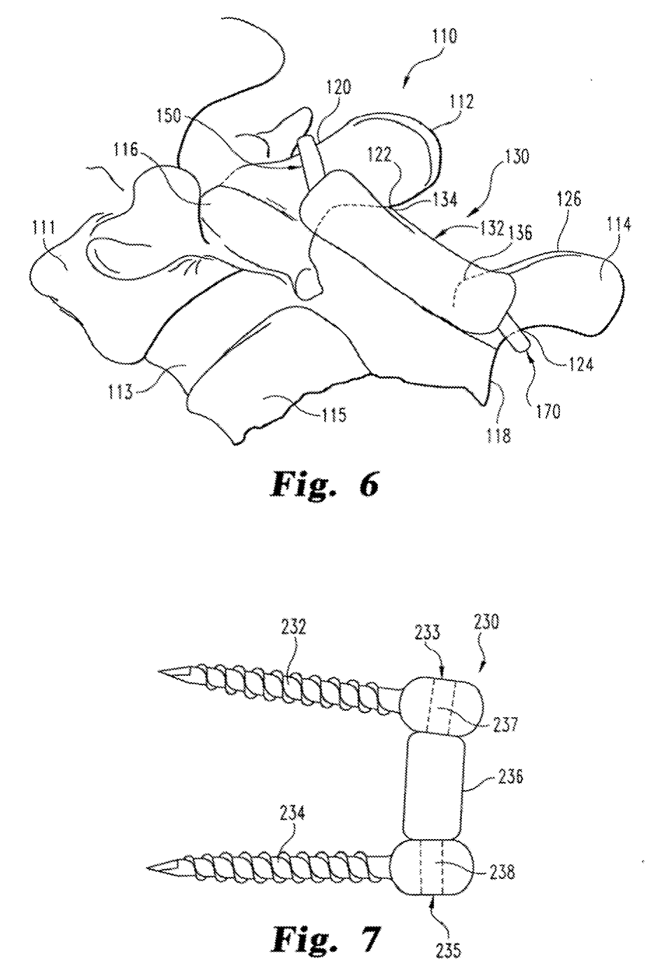

In another preferred embodiment, depicted cross-sectionally in FIG. 2, device 30 comprises an internal structural component 32 contained within an outer sheath 34. In one preferred embodiment, at least one of the internal structural component or the outer sheath comprises an absorbent or adsorbent material having an active agent impregnated therein or adsorbed thereon, and is configured to release the active agent locally after the implant device is implanted in a patient. For example, in one preferred embodiment, inner structural component 32 comprises silicone, which is wrapped in an outer sheath 34 that comprises polyester fabric. In another embodiment, depicted in FIG. 3, device 30 includes an absorbent or adsorbent or biodegradable active agent-delivery layer 36 positioned between internal structural component 32 and the outer sheath 34. In still another embodiment, depicted in FIG. 4, device 30 includes an absorbent or adsorbent or biodegradable active agent-delivery layer 36 positioned on the exterior surface 33 of outer sheath 34. In the embodiment depicted in FIG. 5, device 30 defines aperture 38, and insert 40 is an active agent-delivery component configured to be positioned in the aperture. After the device is implanted, the active agent is released from insert 40 into the area surrounding the device for local administration of the active agent to the affected area.

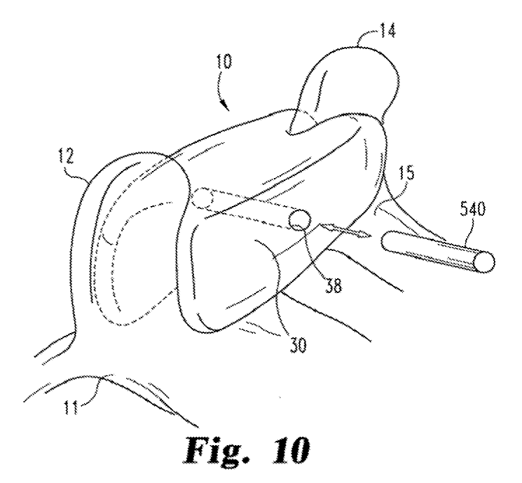

In one embodiment, insert 40 is an active agent-delivery component comprising an absorbent or adsorbent or biodegradable material. In another embodiment, insert 40 is a micromechanical machine 540, as depicted in FIG. 10. Micromechanincal machine 540 is configured to release an active agent in an active mechanical manner rather than a passive manner. For example, micromechanical machine 540 can be a micropump configured to actively release a controlled amount of active agent over time, either as a steady stream or in incremental boluses. Alternatively, micromechanical machine 540 can be configured to release a dose of active agent, for example, by opening a valve or actuating a pump, in response to a signal, such as, for example, a physiological condition sensed by micromechanical machine 540 or a signal received from an ex vivo signaling device. Examples of signals that can be utilized in accordance with the invention include, for example, increased local pressure at the device location, an increased or decreased concentration of a chemical at the device location, increased temperature at the device location, electrical signals, electromagnetic signals, optical signals, magnetic fields and the like.

In FIG. 6 there is shown a spinal column segment 110 including an upper vertebra 111, a lower vertebra 115 and a spinal disc 113 therebetween. The vertebrae 111, 115 and disc 113 comprise a spinal motion segment, it being understood that a spinal motion segment may include multiple vertebral levels. Upper vertebra 111 includes an upper spinous process 112 extending from an upper lamina 116. Lower vertebra 115 includes a lower spinous process 114 extending from a lower lamina 118. The spinous processes 112, 114 and laminae 116, 118 comprise posterior elements of the vertebrae of the spinal motion segment.

A spinal implant assembly 130 is positioned in engagement with the posterior vertebral elements to provide dynamic spinal stabilization. Spinal implant assembly 130 includes a spacer member 132 extending between and contacting adjacent surfaces of spinous processes 112, 114 to limit movement of the spinous processes toward one another as a result of extension of the spinal motion segment. For example, spacer member 132 can include an upper end 134 in contact with inferior surface 122 of spinous process 112, and a lower end 136 in contact with superior surface 126 of spinous process 114. Spacer member 132 can include a body structured to resiliently compress in response to extension of the spinal motion segment, providing resistance to the extension forces and limiting movement of the spinous processes 112, 114 toward one another as spacer member 132 is compressed.

Implant assembly 130 can include an upper engaging member 150 and a lower engaging member 170 extending from spacer member 132. Upper engaging member 150 preferably extends along and contacts a superior surface 120 of spinous process 112, and lower engaging member 170 extends along and contacts an inferior surface 124 of spinous process 114. Engaging members 150, 170, which are preferably tethers, such as cables or straps, thus limit movement of the spinous processes 112, 114 away from one another as a result of flexion of the motion segment. In another embodiment, upper engaging member 150 extends along and contacts a superior surface of upper lamina 116, and lower engaging member 170 extends along and contacts an inferior surface of lower lamina 118. Engaging members 150, 170 can be movably coupled with spacer member 132 to facilitate manipulation of the engaging members 150, 170 and placement over the spinous processes or the spinal lamina.

In this embodiment, device 130, like device 30, can be manufactured of a uniform composition, or can be formed using multiple diverse materials. It is of course understood that spacer member 132 would be formed of one or more compressible materials where it is desired for the implant to be used in an application where it is desirable for spacer member 132 to be compressible. In one preferred embodiment, spacer member 132 has an exterior surface and an active agent-delivery component layer is affixed to at least a portion of said exterior surface. Active agent-delivery layer can be formed on the surface of spacer member 132 in a wide variety of ways known in the art. Similarly, spacer member 132, like device 30, can have alternative structures as represented cross-sectionally in FIGS. 2-4, and can include the aperture/insert configuration as represented in FIG. 5.

Some implant assembly embodiments contemplated by the invention utilize a connecting member (not shown) connected to engaging members 150, 170 that extends through the body of spacer member 132 so that it is not exposed to the anatomy outside and adjacent spacer member 132 when implanted. This arrangement avoids exposure of the connecting member to the spinal foramen and neural elements, for example. The connection of the connecting member to the engaging members at locations along the respective arms 142, 144, also avoids exposure to the foramen. The connecting member can be positioned through one or more passages formed in the spacer member, or the spacer member can be over-molded about the connecting member. Various forms for the connecting members are contemplated, including cables, wires, sutures, cords, bands, belts, rigid links or rods, and flexible links or rods, for example. The present invention contemplates that the connecting members and/or the engaging members can have an active agent-delivery component associated therewith, in addition to or instead of having an active agent-delivery component associated with spacer member 132. For example, these elements can be made of woven or otherwise porous structural materials and have an active agent impregnated therein, or these elements can have an active agent-delivery layer provided therein or thereon, which can be an absorbable or biodegradable material having an active agent impregnated therein, or can be a material having an active agent adsorbed thereto.

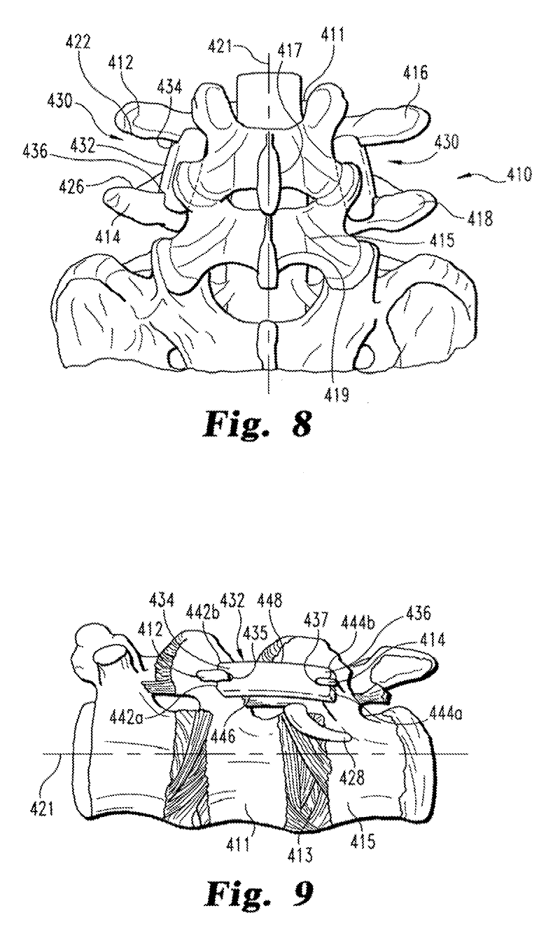

In another embodiment of the invention, depicted in FIG. 7, an anchor-based spinal stabilization or spinal fixation device, such as, for example, a pedicle screw-based system 230 is provided. System 230 includes first anchor (also referred to herein as a pedicle screw in relation to some embodiments) 232 configured to be anchored in a first vertebra (not shown) and second anchor 234 (also referred to herein as a pedicle screw in relation to some embodiments) configured to be anchored in a second vertebra (not shown) adjacent the first vertebra. System 230 also includes spacer element 236 configured for placement between head portion 233 of first anchor 232 and head portion 235 of second anchor 234.

Spacer member 236 can have many or all of the same attributes as the spacer members discussed above with respect to an interspinous dynamic stabilization device. As will be appreciated by a person skilled in the art, once anchors 232, 234 are rigidly connected to adjacent vertebrae in a patient's spine, flexion, extension, bending or twisting of the spine will cause anchors 232, 234 to move relative to one another. Where spacer 236 comprises a compressible material, extension of the patient's spine can be limited by placing spacer 236 between heads 233, 235 of anchors 232, 234. In an embodiment in which spacer 236 comprises a compressible, absorbent material with an active agent impregnated therein, compression can cause release of the active agent as in dynamic stabilization devices described above.

In certain embodiments, spacer 236 defines a channel therethrough (not shown) for receiving a tether, rod or other structural component (not shown). For example, the tether, rod or other structure can pass through the channel and pass through apertures 237, 238 formed in heads 233, 235, respectively, and can be attached thereto using means known in the art to provide spinal stabilization or spinal fixation functionality. Alternatively, spacer 236 can be enveloped in a sheath (not shown) that is configured to envelope spacer 236 and pass through apertures in heads 233, 235.

In this embodiment, spacer 236, like device 30, can be manufactured of a uniform composition, or can be formed using multiple diverse materials. It is of course understood that spacer 236 would be formed of one or more compressible materials where it is desired for the implant to be used in an application where it is desirable for spacer 236 to be compressible. In one preferred embodiment, spacer 236 has an exterior surface and an active agent-delivery component layer is affixed to at least a portion of said exterior surface. Active agent-delivery layer can be formed on the surface of spacer 236 in a wide variety of ways known in the art. Similarly, spacer 236, like device 30, can have alternative structures as represented cross-sectionally in FIGS. 2-4, and can include the aperture/insert configuration as represented in FIG. 5.

In FIG. 8 there is shown a spinal column segment 410 including an upper vertebra 411, a lower vertebra 415 and a spinal disc 413 therebetween along a central axis 421 of the spinal column. The vertebrae 411, 415 and disc 413 comprise a spinal motion segment, it being understood that a spinal motion segment may include multiple vertebral levels. Upper vertebra 411 includes a first upper transverse process 412 and a second upper transverse process 416. Lower vertebra 415 includes a first lower transverse process 414 and a second lower transverse process 418. The transverse processes 412, 414, 416, 418 comprise posterior elements of the vertebrae of the spinal motion segment along with the spinous processes 417, 419, facets, pedicles and other posterior structures of each vertebrae 411, 415.

A spinal implant 430 is positioned in engagement with the posterior vertebral elements to provide dynamic spinal stabilization. Spinal implant 430 includes a spacer member 432 extending between and contacting adjacent surfaces of transverse processes 412, 414 to limit movement of the spinous processes toward one another as a result of extension of the spinal motion segment. For example, spacer member 432 can include an upper end 434 in contact with inferior surface 422 of transverse process 412, and a lower end 436 in contact with superior surface 426 of transverse process 414. Spacer member 432 can include a body structured to resiliently compress in response to extension of the spinal extension, providing resistance to the extension forces and limiting movement of the transverse processes 412, 414 toward one another as spacer member 432 is compressed.

Spacer member 432, like device 30 and spacer member 130, can be manufactured of a uniform composition, or can be formed using multiple diverse materials. It is of course understood that spacer member 432 would be formed of one or more compressible materials where it is desired for the device to be used in an application where it is desirable for spacer member 432 to be compressible. In one preferred embodiment, spacer member 432 has an exterior surface and an active agent-delivery component layer is affixed to at least a portion of said exterior surface. Active agent-delivery layer can be formed on the surface of spacer member 432 in a wide variety of ways known in the art. Similarly, spacer member 432, like device 30 and spacer member 130, can have alternative structures as represented cross-sectionally in FIGS. 2-4, and can include the aperture/insert configuration as represented in FIG. 5.

FIG. 8 further shows a second spinal implant 430 on the other side of central axis 421 of the spinal column. The second spacer member 432 can be structured like the other implant 430, and is configured to extend between and contact adjacent surfaces of transverse processes 416, 418 to limit movement of the spinous processes toward one another as a result of extension of the spinal motion segment. The implants 430 work bi-laterally to provide bi-lateral stabilization of spinal column segment 410. Additional implants 430 may be provided at one or more additional vertebral levels for multi-level stabilization procedures. It is further contemplated that implants 430 may be employed to uni-laterally stabilize one or more vertebral levels. The spinal implants, either alone or in combination, can function to distract the spinal space and/or the spinal foramen to relieve nerve root pressure, decompress spinal elements. The implants provide overall stability while maintaining motion capabilities of the spinal motion segment.

As further shown in FIG. 9, spacer member 432 includes a pair of upper arms 442a and 442b and a pair of lower arms 444a and 444b. Each of the arm pairs includes an anterior arm 442a, 444a and posterior arm 442b, 444b. Upper arms 442a and 4442b define a concavely curved upper surface 435 therebetween, and lower arms 444a and 444b define a concavely curved lower surface 437 therebetween. The concavely curved surfaces 435, 437 can conform generally to or be conformable to the surface of the transverse process against which the surface is positioned. Arms 442a, 442b, 444a, 444b extend along opposite sides of and receive the respective transverse process 412, 414 to resist dislodgement of spacer member 432 from its positioning between transverse processes 412, 414. In its implanted orientation, spacer member 432 includes an anteriorly oriented surface 446 and a posteriorly oriented surface 448. Anteriorly oriented surface 446 can include a concave curvature to fit over the exiting nerve root 428 and prevent or avoid any impingement thereof. Posteriorly oriented surface 448 can be convexly curved as illustrated, or can include a concave curvature, or it can be linear in form. In the illustrated embodiment, anterior arms 442a, 444a have a thickness that is less than the thickness of the posterior arms 442b, 444b. The reduced thickness limits the amount of spacer material in the area where nerve root 428 exits the spinal foramen, increasing the space available for nerve root 428 to pass.

An engaging member (not shown) can be employed to secure the spacer member in place. The engaging member can be in the form of a tether, cord, wire, cable, suture, band, strap, belt, or other suitable structure for manipulation and securement to one or more posterior vertebral elements. The engaging member can be wrapped or positioned around posterior vertebral elements and then maintained in position with a crimp or other suitable fastener. Furthermore, the engaging member can be coupled to the spacer member in any suitable manner. In one embodiment, the engaging member is movably coupled to the spacer member. The engaging member can be integrally formed with the spacer member, or can be attached by a fastener, suture, anchor, cable, link, over-molding, thermal welding or bonding, adhesive bonding, three dimensional weaving or braiding, screws, staples, pins, tacks, rivet fixation or other suitable connection. The spacer member can be provided with ears, eyelets, recesses or other suitable structure to facilitate engagement of the engaging member to the spacer member. The engaging member may be employed in spinal stabilization procedures where it is desired to limit spinal flexion by, for example, wrapping the engaging member about the superior surface of the upper transverse process and the inferior surface of the lower transverse process. The engaging member may alternatively be employed as a retention mechanism to maintain the spacer member in position between the transverse processes.

The engaging member can be secured to the spacer member either before or after the spacing member is placed between the transverse processes. The engaging member can be engaged to other engaging members of other implant assemblies or to other implants engaged to the spinal column in the surgical procedure. The present invention contemplates that the engaging members can have an active agent-delivery component associated therewith, in addition to or instead of having an active agent-delivery component associated with spacer 430. For example, these elements can be made of woven or otherwise porous structural materials and have an active agent impregnated therein, or these elements can have an active agent-delivery layer provided therein or thereon, which can be an absorbable or biodegradable material having an active agent impregnated therein, or can be a material having an active agent adsorbed thereto.

The engaging members described herein can be made from any one or combinations of biocompatible material, including synthetic or natural autograft, allograft or xenograft tissues, and can be resorbable or non-resorbable nature. Examples of tissue materials include hard tissues, connective tissues, demineralized bone matrix and combinations thereof. Further examples of resorbable materials are polylactide, polyglycolide, tyrosine-derived polycarbonate, polyanhydride, polyorthoester, polyphosphazene, calcium phosphate, hydroxyapatite, bioactive glass, and combinations thereof. Further examples of non-resorbable materials are carbon-reinforced polymer composites, silicone, PEEK, shape-memory alloys, titanium, titanium alloys, cobalt chrome alloys, stainless steel, and combinations thereof.

As will be appreciated by a person of ordinary skill in the art in view of the descriptions herein, the present invention provides in one aspect a posterior spinal fixation device or dynamic spinal stabilization device that includes an active agent-delivery component. The active agent-delivery component has an active agent impregnated therein or adsorbed thereon or otherwise contained therein and is configured to release the active agent locally after the device is implanted in a patient. In one preferred embodiment, the device is a dynamic stabilization device configured for placement between adjacent spinous processes, between adjacent transverse processes or between other posterior vertebral elements. In one embodiment, the device is an inter-spinous process dynamic stabilization device. In another embodiment, the device is an inter-transverse process dynamic stabilization device. In yet another embodiment, the device is an anchor-based stabilization or fixation system.

In one form of the invention, an inventive device comprises at least one structural component configured to provide spinal stabilization, and at least a portion of at least one of the structural components has the active agent impregnated therein or adsorbed thereon. For example, one preferred device comprises an internal structural component contained within an outer sheath, wherein the outer sheath includes an absorbent or adsorbent or biodegradable material having the active agent impregnated therein or adsorbed thereon. The active agent can be selected, for example, from the group consisting of an anabolic agent, an anti-coagulant, an anti-infective agent, an anti-inflammatory agent, an anti-neoplastic agent, an anti-pyretic agent, an analgesic agent, an anti-spasmodic agent, an anti-thrombotic agent, an antihistamine, a biological, a bone morphogenetic protein, a diagnostic agent, a neuromuscular drug, a nutritional agent, a vasodilator, and a pro-drug.

The amount of active agent incorporated in the device can vary depending on the particular active agent used, the desired therapeutic effect, and the time-span over which delivery of the active agent is desired. A variety of devices in a variety of sizes and shapes can be fashioned according to the present invention to include the active agent-delivery component, and which are intended to provide dosage regimes for therapy of a variety of conditions. The upper and lower limits will depend on the activity of the active agent and the time span of its release from the device desired in a particular application.

In another form of the invention, an inventive device comprises at least one structural component configured to provide spinal stabilization and at least one active agent-delivery component retained by the structural component. In one preferred embedment, the device includes an internal structural component positioned within an outer sheath, and the active agent-delivery component comprises an absorbent or adsorbent or biodegradable layer positioned between the internal structural component and the outer sheath. In another preferred embodiment, the device has an exterior surface and the active agent-delivery component comprises an active agent-delivery layer affixed to at least a portion of said exterior surface. In yet another preferred embodiment, the structural component defines at least one aperture and the active agent-delivery component is an insert configured to be positioned in the aperture. The insert in certain preferred embodiments comprises a micromechanical machine.

In one preferred embodiment, the active agent-delivery component comprises an elastic material having the active agent absorbed therein or adsorbed thereon. The device, after implantation of device, releases the active agent, preferably in a sustained release manner, or in a controllable or semi-controllable manner. For example, the device can be configured such that, after implantation of the device, a dose of the active agent is caused to be released or released at an increased rate by compressing the active agent-delivery component, or by stretching the component, or by applying a torque to the component. In one preferred embodiment, the device is an inter-spinous process dynamic stabilization device, and the device is configured such that, after implantation, compressive pressure, stretching or torque is exerted upon the active agent-delivery component by vertebral movement as a result of extension of the spinal column, flexion of the spinal column, bending of the spinal column or rotation of the spinal column. In another preferred embodiment, the device is an inter-transverse process dynamic stabilization device, and the device is configured such that, after implantation, compression, stretching or torque is exerted upon the active agent-delivery component by vertebral movement as a result of extension of the spinal column, flexion of the spinal column, bending of the spinal column or rotation of the spinal column. In yet another embodiment, the device is an anchor-based fixation or stabilization system:

In another form of the invention, there is provided an orthopedic implant device comprising an active agent-delivery component, wherein the active agent-delivery component comprises an elastic material having the active agent absorbed therein or adsorbed thereon, wherein the device is configured to release the active agent locally after the device is implanted in a patient, and wherein the device is configured such that a dose of the active agent is caused to be released or released at an increased rate by (a) exerting compressive pressure upon the active agent-delivery component, (b) stretching the component, or (c) applying a torque to the component. In one embodiment, the device includes an internal structural component positioned within an outer sheath, and the outer sheath comprises an absorbent or adsorbent or biodegradable material having the active agent impregnated therein or adsorbed thereon. In another embodiment, the device includes an internal structural component positioned within an outer sheath, and the active agent-delivery component comprises an absorbent or adsorbent or biodegradable layer positioned between the internal structural component and the outer sheath. In yet another embodiment, the device has an exterior surface and the active agent-delivery component comprises an active agent-delivery layer affixed to at least a portion of the exterior surface. In still another embodiment, the at least one structural component defines at least one aperture and the active agent-delivery component is an insert configured to be positioned in the aperture.

While the invention has been illustrated and described in detail in the drawings and foregoing description, the same is to be considered illustrative and not restrictive in character, it being understood that only selected embodiments have been shown and described and that all changes, equivalents, and modifications that come within the scope of the inventions described herein or defined by the following claims are desired to be protected. Any theory, mechanism of operation, proof, or finding stated herein is meant to further enhance understanding of the present invention and is not intended to limit the present invention in any way to such theory, mechanism of operation, proof, or finding. Further, any U.S. patent or pending U.S. patent application Publication cited herein is incorporated herein by reference in its entirety. In reading the claims, words such as “a”, “an”, “at least on”, and “at least a portion” are not intended to limit the claims to only one item unless specifically stated to the contrary. Further, when the language “at least a portion” and/or “a portion” is used, the claims may include a portion and/or the entire item unless specifically stated to the contrary.

What is claimed is:

1. A method for delivering an active agent to a patient at a location adjacent an orthopedic implant device, comprising:
providing at least one flexible structural component configured to provide spinal stabilization, the structural component having a bore defined therein;
inserting an active agent-delivery component into the bore so as to be retained therein, wherein the active agent-delivery component has an active agent absorbed therein, adsorbed thereon, or otherwise contained therein, and is configured to release the active agent locally after the device is implanted in a patient;
surgically implanting the structural component in a posterior spinal location;
wherein a combination of the structural component and the insert is configured to respond to the application of force to an exterior of the structural component, after the device is implanted, by releasing the active agent therefrom;
wherein the application of force to the exterior of the structural component directly causes the active agent to be passively released from the insert.

2. The method of claim 1 wherein the surgically implanting comprises surgically implanting the device between an upper spinous process and a lower spinous process such that the bore extends through a sagittal plane defined by the upper and lower spinous processes.

3. The method of claim 1 wherein inserting an active agent-delivery component into the bore comprises inserting the active agent-delivery component into the bore such that the bore entirely surrounds a cross-sectional perimeter of the active agent-delivery component.

4. A dynamic spinal stabilization device comprising:
at least one flexible structural component configured to provide spinal stabilization;
an insert disposed in the bore and having an active agent absorbed therein, adsorbed thereon, or otherwise contained therein; the insert configured to release the active agent locally after the device is implanted in a patient;
wherein a combination of the structural component and the insert is configured to respond to the application of force to an exterior of the structural component by releasing the active agent therefrom;
wherein the application of force to the exterior of the structural component directly causes the active agent to be passively released from the insert.

5. The device of claim 4 wherein said device is configured for placement between adjacent spinous processes.

6. The device of claim 4 wherein said active agent is selected from the group consisting of an anabolic agent, an anti-coagulant, an anti-infective agent, an anti-inflammatory agent, an anti-neoplastic agent, an anti-pyretic agent, an analgesic agent, an anti-spasmodic agent, an anti-thrombotic agent, an antihistamine, a biological, a bone morphogenetic protein, a diagnostic agent, a neuromuscular drug, a nutritional agent, a vasodilator, and a pro-drug.

7. The device of claim 4 wherein said device is configured such that, after implantation, a dose of said active agent is caused to be released or released at an increased rate by compressing said active agent-delivery component, or by stretching said component, or by applying a torque to said structural component.

8. The device of claim 4 wherein when the device is disposed between an upper spinous process and a lower spinous process, the bore extends through a sagittal plane defined by the upper and lower spinous processes.

9. The device of claim 4 wherein at least a portion of the bore surrounds a cross-sectional perimeter of the insert disposed therein.

10. An orthopedic implant device comprising:
a flexible structural component having a bore defined therein;
an active agent-delivery component insert received in the bore, wherein said insert comprises an elastic material having said active agent absorbed therein or adsorbed thereon;
wherein a combination of the structural component and the insert is configured to respond to the application of force to an exterior of the structural component, after the device is implanted, by releasing said active agent locally.

11. The device of claim 10 wherein said implant is a dynamic stabilization device configured for placement between adjacent spinous processes.

12. The device of claim 10 wherein said insert is configured to release said active agent in a sustained release manner after the device is implanted in a patient.

13. The device of claim 10 wherein when the device is disposed between an upper spinous process and a lower spinous process, the bore extends through a sagittal plane defined by the upper and lower spinous processes.
